(19) United States
(12) Patent Application Publication
(10) Pub. No.: US 2007/0141008 A1
(43) Pub. Date: Jun. 21, 2007

(54) Cosmetic remover composition

(76) Inventor: Dennis R. Jones, Chesapeake, VA (US)

Correspondence Address: KEVIN W. GRIERSON;WILLCOX & SAVAGE, P.C., ONE COMMERCIAL PLACE, SUITE 1800, NORFOLK, VA, 23510, US

(21) Appl. No.: 11/640,627

(22) Filed: Dec. 18, 2006

Related U.S. Application Data
(60) Provisional application No. 60/751,018, filed on Dec. 16, 2005.

(51) Int. Cl. A61K 8/89

(52) U.S. Cl. 424/70.12

(57) ABSTRACT

A cosmetic removal composition suitable for removing “permanent” or long lasting color cosmetics is described. The composition comprises at least one silicone crosspolymer with solvent properties.

### CLAIM OF PRIORITY

This patent application claims the benefit of provisional patent application number 60/751,018, filed Dec. 16, 2005.

### FIELD OF INVENTION

The present invention relates to a new cosmetic remover composition, principally a makeup and “permanent” lip coloring remover.

### BACKGROUND OF THE INVENTION

Two types of long-lasting or “permanent” makeup are particularly difficult to remove: “Permanent” lip colors, which are actually paints or shellacs that seal the color under a polymer film, and liquid face makeups, which are generally silicone-in-silicone emulsions. Although both types of products do not smudge or come off easily, requiring less maintenance, both are also quite difficult to remove when the user wants to take them off. Most current cosmetic remover compositions are ineffective at removing such “permanent” cosmetics. A cosmetic remover composition that easily removes either permanent lip colors or permanent face makeups, or both, is highly desirable, particularly if such a composition is gentle to the skin.

### DESCRIPTION OF PRIOR ART

U.S. Pat. No. 5,034,489 to Wengrovius, et. al., and assigned to General Electric, describe a crosslinkable silicone copolymer having chemically combined tetraorganodisilyloxy groups with the same or different monovalent C(1-4) hydrocarbon radicals and C(1-14) hydrocarbon radials substituted with monovalent radicals inert during equilibration wherein the formulation is useful as a waterproofing agent.

U.S. Pat. No. 6,908,621 to Jose, et. al., and assigned to Revlon, describes an anhydrous silicone compatible organic pigmented oil cosmetic composition consisting of monoester, diester, triester, and mixtures thereof; and at least two silicones and at least one synthetic film forming polymer that is a solid or liquid at room temperature and which is a lipstick, blush, eyeshadow, foundation, or concealer.

U.S. Pat. No. 5,106,609 to Bolich, et. al., and assigned to Procter & Gamble, describe a cosmetic composition comprising from about 0.1% to about 10.0% by weight of the cosmetic composition of a hydrophobically modified nonionic water-soluble polymer wherein said cosmetic compositions comprise no more than about 1.0% of water-soluble surfactants.

U.S. Pat. No. 5,997,853 to Bolich, et. al., and assigned to Procter & Gamble, describe a copolymer hair care composition having an average molecular weight of from about 10,000 to about 1,000,000, prepared from a polydlmethylsiloxane macromonomer having a weight average molecular weight from about 1000 to about 50,000, and a component selected from the group consisting of t-butyl acrylate and mixtures of t-butyl acrylate and acrylic acid, wherein said copolymer comprises up to about 98 weight percent of t-butyl acrylate, up to about 98 weight percent of acrylic acid when used, and about 0.01 to about 50 weight percent of said polydimethylsiloxane macromonomer, and a carrier for said copolymer.

U.S. Patent Publication No. 2003/0195316 to Stark, Kurt, and assigned to Wacker Polymer Systems GmbH & Co. Kg., describes an organic waterproof silicone copolymer useful as a release agent for cosmetics and adhesives.

U.S. Patent Publication No. 20030105169A1 to Lennon, Paula, and assigned to L'Oreal, describe a composition in the form of an oil-in-water emulsion comprising an oily phase dispersed in an aqueous phase, wherein the aqueous phase comprises particles of uncrosslinked silicone copolymer and at least one amphiphilic surfactant capable of forming liquid crystals useful in a cosmetic or dermatological composition wherein the composition constitutes a product for caring for, treating, protecting, cleaning, removing make-up from and/or making-up keratinous substances wherein the keratinous substance is the skin. It also includes a method for cleaning and/or removing make-up from the skin and/or eyes, comprising applying the composition to made-up skin and/or eyes.

U.S. Patent Publication No. 2003/0068291A1 to Decoster, et. al., and assigned to L'Oreal, describe a cosmetically acceptable medium, a thickener and an aqueous emulsion comprising at least one silicone copolymer wherein the silicone copolymer is essentially non-crosslinked and at least one additive is chosen from fragrances, nacreous agents, preserving agents, silicone sunscreens, non-silicone sunscreens, vitamins, provitamins, amphoteric, anionic and nonionic polymers, proteins, protein hydrolysates, 18-methyleicosanoic acid, hydroxy acids, panthenol, volatile and non-volatile, cyclic and linear and crosslinked, modified and non-modified silicones, ceramides, pseudoceramides, and plant, animal, mineral and synthetic oils and a process for washing or treating hair, skin, eyelashes, eyebrows, nails, lips and scalp.

U.S. Pat. No. 6,451,298 to Decoster, et. al., and assigned to L'Oreal, describe a cosmetic composition comprising at least one silicone copolymer in the presence of a catalyst of at least one cationic polymer wherein the combination can give cosmetic properties, such as at least one of smoothness, lightness, and softness, without the phenomenon of regreasing keratin fibers. These compositions can be used for washing and/or conditioning a keratin material, such as the hair or the skin.

U.S. Pat. No. 5,972,095 to Grave, et. al., and assigned to Maybelline Cosmetics Corp., describe a fast-drying nail enamel composition which comprises a solvent solution of plasticized film-forming cellulose derivative, vinyl-silicone graft comprising a silicone polymer segment and a vinyl polymer segment and a dimethicone anti-foaming agent.

U.S. Pat. No. 5,667,765 to Hansenne, et. al., and assigned to L'Oreal, describes a topically applicable sunscreen/cosmetic composition adopted for the photoprotection of human skin and/or hair. The sunscreen/cosmetic composition further comprising at least one derivative of cinnamic, salicylic, camphor, triazine, benzophenone, dibenzoylmethane, β,β-diphenylacrylate, p-aminobenzoic acid and sunscreen polymer or silicone.

U.S. Pat. No. 5,527,488 to Groh, David, and assigned to Amway Corp., describes a high viscosity anhydrous eye makeup remover composition for application to the skin on and around the eyelid consisting essentially of a non-irritating oil base, glyceryl trioctanoate, cetyl octanoate, caprylic capric triglycerides, polyethylene and ethylene vinyl acetate copolymers. The weight proportion of the polyethylene to the ethylene vinyl acetate copolymer is in the range of about 0.67:1 to about 2.0: 1, wherein the composition has a viscosity in the range of 7,000 to 15,000 poise.

U.S. Patent Publication No. 2002/0098209A1 to Najdek, et. al., and assigned to Estee Lauder (E-L Management Corp.), describe a dual phase liquid cosmetic or pharmaceutical composition comprising an aqueous phase and an oil phase. The composition includes a volatile oil of both a volatile silicone and a volatile isoparaffm wherein the volatile silicone is cyclomethicone and the volatile isoparaffm is a C16 isoparaffm and the non-volatile silicone oil is dimethicone.

U.S. Pat. No. 5,871,758 to Nagy, et. al., and assigned to Estee Lauder (E-L Management Corp.), describe a dual phase liquid cosmetic or pharmaceutical composition containing as a demixing agent an effective amount of a quaternary nitrogen-containing ether.

U.S. Pat. No. 5,165,917 to Zabotto, et. al., and assigned to L'Oreal, describe a non-foaming cosmetic composition suitable for removal of both waterproof eye makeup and non-waterproof eye makeup comprising two separate phases comprising at least one surfactant selected from the group consisting of anionic surfactants, nonionic surfactants and amphoretic surfactants, and at least one cosmetic oil wherein said oil is selected from the group consisting of Vaseline oil, isohexadecane, jojoba oil, safflower oil, silicone oil, and synthetic oil wherein silicone oil is present.

U.S. Pat. No. 4,806,572 to Kellett, George, and assigned to Creative Products Resources Associates Ltd., describe a makeup remover pad comprising a resilient, open-celled, hydrophilic polyurethane foam matrix, incorporating about 25-75% water, water-insoluble liquid emollient oil and surfactant effective to stabilize the aqueous phase so that it is released from the foam matrix as a homogenous emulsion when the pad is applied to skin. The aqueous phase contains no natural or synthetic wax and the emollient oil comprises mineral oil, silicone fluid or mixtures and an organic emollient ester.

U.S. Patent Publication No. 2004/0126349A1 to Anderson, et. al., and assigned to Avon, describe a method of decreasing the amount of sebum on a person's hair or scalp comprising applying a non-emulsion water-based composition comprising a crosslinked silicone elastomer to said hair or scalp.

U.S. Pat. No. 6,238,656 to Morita, et. al., and assigned to Dow Corning, describe a method of treating hair or skin comprising silicone oil in water emulsion consisting essentially of crosslinked silicone particles, crosslinked silicone particles being dispersed in water and a non-crosslinkable silicone oil, a surface active agent, a catalyst, and conducting a crosslinking reaction.

U.S. Pat. No. 4,696,974 to Sule, et. al., and assigned to Ceskoslevenska Akademie Ved, describe a hydrophilic silicone composite comprising a crosslinked silicone polymer matrix, and a powdered hydrogel filler selected from the group consisting of cross-linked polymers and copolymers of glycol monomethacrylates, polyol monomethacrylates, dihydroxyester monomethacrylates, acrylamide, methacrylamide, N-substituted acrylamide, N-substituted methacrylamide, N,N-disubstituted acrylamide, N,N-disubstituted methacrylamide, and multiblock copolymers of acrylonitrile with acrylamide and/or acrylic acid and useful for biomedical implants, contact lenses etc.

U.S. Patent No. 6,585,985 to Sakuta, Koji, and assigned to Shin-Etsu Chemical Co. Ltd., describes a lipstick composition which comprises a crosslinked organopolysiloxane compound having at least one hydrophilic group, crosslinked organopolysiloxane, an oily compound selected from the group consisting of liquid hydrocarbon compounds, esters, higher fatty acids, higher alcohols and liquid silicone compounds, a pigment and the balance being additives permissible as an ingredient of a cosmetic preparation.

WIPO Publication WO 04066972A1 to Paul, et. al. and assigned to Unilever PLC, describes a hair cleansing cosmetic composition comprising a cationised fatty alkyl silicone copolymer, a cationised dimethiconol complex and a cationised high viscosity lower alkyl silicone an active detergent.

European Patent Publication EP0540357B1 to Jones, et. al. and assigned to Croda International PLC, describes a protein-silicone copolymer useful in cosmetic formulations that is covalently bonded to amino groups of protein and at least some of the silicone components forms cross-links between different protein chains with the protein component forming from 5% to 98% by weight of the copolymer.

European Patent Publication EP1325729A3 to Lennon, Paula, assigned to L'Oreal, describes a cosmetic composition containing a silicone copolymer and an AMPS polymer.

European Patent Publication EP1325729A3 to Lennon, Paula, and assigned to L'Oreal, describe a cosmetic oil-in-water emulsion containing a silicone copolymer.

European Patent Publication EP0446156A1 to Jorda, et. al., and assigned to Rhone-Poulenc Chimie, describes a hydrolytically degradable polyester-silicone jointly crosslinked copolymer according to the invention can be employed in particular as a matrix containing an active principle with a view to the controlled release of the active principle by simple erosion of this matrix by hydrolysis.

WIPO Publication WO04020526A1 to Morita, et. al. and assigned to Dow Corning, describes cosmetic ingredients of an aqueous suspension of crosslinked silicone particles, N-acyl-N-hydrocarbyltaurine and/or a salt thereof and water therefore a cosmetic ingredient consisting of the suspension or the emulsion. The suspension and the emulsion are characterized by excellent stability, decreased load on the environment, and little influence on the human body, and exhibiting excellent cosmetic functions such as moisturizing and smoothening.

WIPO Publication WO03053372A1 to Maxon, et. al. and assigned to Dow Corning, describes a composition comprising a water-in-oil emulsion having an aqueous phase dispersed in a continuous oil phase. The continuous oil phase comprising a linear silicone polyether, a crosslinked silicone elastorner, and a nonionic organic emulsifier, the emulsion being free of silicone elastomers wherein the composition active ingredient is selected form the group consisting of Vitamin B 1, Vitamin B2, Vitamin B6, Vitamin B 12, niacin, folic acid, biotin, pantothenic acid, Vitamin E, Tocopherol, cc-Tocopherol, P-Tocopherol, y-Tocopherol, A-Tocopherol, Tocophersolan, Tocopheryl Acetate, Tocopheryl Pahnitate, Tocopheryl Linoleate, Tocopheryl Nicotinate, Tocopheryl Succinate, and mixtures thereof.

European Patent Publication EP1 136064A3 to Anderson, Glen, and assigned to Avon, describes a cosmetic composition comprising a crosslinked silicon elastomer selected from the group consisting of: dimethicone crosspolymer; organopolysiloxane; polysilicone-11; and dimethicone/vinyl dimethicone crosspolymer; and mixtures thereof. And a secondary component selected from the group consisting of an estrogen synthetase stimulating compound, a collagen enhancing agent; a phytoestrogen and mixtures thereof.

European Patent Publication EP0709422A3 to Friebe, Robert, and assigned to Bayer AG, describes an activator for depolymerising crosslinked polysiloxanes such as phosphoric acid and/or mono- and/or di-esters thereof. Also claimed is the same activator, together with auxiliary substance(s), for the depolymerisation of crosslinked and/or uncrosslinked polysiloxanes; a process for the deactivation of crosslinked, optional filler-containing. and/or uncrosslinked polysiloxanes by treatment with the above activator.

### SUMMARY OF INVENTION

One object of the present invention is to provide one solution to the problem of removing a cosmetic, makeup, and/or permanent, semi-permanent or long-lasting lip color or lipstick, from the skin by offering a composition able to remove long lasting, waterproof, transfer resistant and non-waterproof cosmetics for eyes, lips and faces in a particularly effective manner, most preferably with a single application of the removing composition.

The composition of the present invention includes a cosmetic remover comprising a unique silicon crosspolymer with solvent properties that functions in two ways: on permanent lip colorings, it dissolves the color-protecting polymeric film; for silicone-in-silicone emulsion based permanent makeup, it solublizes the makeup.

Preferably the invention is a cosmetic remover useful in removing “smear-resistant” or “transfer-resistant” cosmetic films initially adhered to the skin or mucous membranes.

Preferably the invention enhances the user's natural skin patina.

Preferably the invention is dispensed in a gel, cream or semi-liquid state.

Additionally the invention may be used for removing waterproof and non-waterproof cosmetics from the skin and mucous membranes.

Additionally the invention may be used for removing waterproof and non-waterproof cosmetics from fabrics such as carpets, clothing and upholstery.

### DETAILED DESCRIPTION OF THE INVENTION

The present invention relates to a cosmetic removal composition that functions in two ways: (1) by dissolving the polymer film for permanent lip colorings, and (2) by resolublizing the silicone based compositions of permanent makeup.

In a preferred embodiment, there is additionally a deposition of agents, such as essential fatty acids, found in interstitial fluids of the skin and ceramides found in the collagen layer after removal is completed. Silicone crosslinked polymers are known to provide a light, silky feel on skin and hair.

It is well known that silicones constitute active beneficial ingredients for various cosmetic formulations, its selection and form of incorporation in various cosmetic formulations is important to achieve the desired stability, compatibility with various other ingredients, and importantly, the effective deposition of the silicones onto substrates as beneficial agents. The cosmetic composition can be provided in variety of formulations, in particular, cleansing or health care formulations for cosmetic applications.

A working example of the composition of the present invention is detailed in Table 1 below. The cosmetic remover composition of the invention may be provided in gel or liquid form, and may be sold in a tube, roll-on dispenser, tub or pump dispenser.

TABLE 1
Chemical composition of cosmetic remover

| | | Name: CTFA or | | Formula | |
|---|---|---|---|---|---|
| | Chemical | USP (Vendor trade | | Qty, | |
| Phase | ID Code | name) | Formula. % | lbs | Function |
| A | Cyclomethicane | Cyclopentasiloxane | 89.000 | | Silicon elastomer |
| | (generic) | (and) | | | moisture barrier |
| | Dow 344, | Dimethicone/Vinyl | | | and makeup |
| | 345 | Dimethicone | | | remover long |
| | | Crosspolymer | | | lasting makeup and |
| | | | | | lipstick is silicon |
| | | | | | based, and is |
| | | | | | dissolved by this |
| | | | | | silicon polymer |
| B | | Dioctyldodecyl | 0.500 | | Essential fatty |
| | | Dimer Dilinoleate | | | acids nutritive to |
| | | (and) Omega-6- | | | the skin, found in |
| | | linoleic acid | | | the interstitial fluid |
| B | | Ceramide 3 | 0.500 | | Skin identical |
| | | | | | ceramides found |
| | | | | | also in the collagen |
| | | | | | layer |
| B | | Octyl cocoate | 10.00 | | Silicon compatible |
| | | | | | emollient and skin |
| | | | | | softner |

The composition of phase A is comprised of two compounds: (1) a specialty silicon cross polymer with solvent properties (dimethicone/vinyl dimethicone copolymer) mutually soluble in virtually all of the various combinations of ingredients used in today's commercial transfer-resistant lip colorings and face and eye cosmetics; and (2) a cyclic dimethyl polysiloxane (here which helps to provide a useable consistency for the cosmetic remover, and also functions as an emollient.

The present invention removes the lip color by dissolving or solublizing the color containing semi-permanent film adhering to the skin or mucous membranes. This film is primarily composed of synthetic polymers such as polyvinylpyrollicone/hexadecane copolymer, PPG-51 SMDI copolymer, vinyl acetate, acrylic acid esters and a barrier forming silicones such as trimethylsiloxy silicate. The present invention removes permanent makeup based on silicone in silicone emulsion by solublizing the makeup.

In a preferred embodiment, the present invention provides a polymer and ester combination comprised of a unique silicon crosspolymer with solvent properties combined with a cyclic dimethyl polysiloxane and, optionally, a compatible and soluble emollient oil (such as octyl cocoate), which is an ester derived from coconut oil. The silicone crosspolymer's uniqueness comes from its ability to codissolve the many film-forming components of the various transfer-resistant commercial products. While many other silicone derivatives can also solublize the various components of the semi permanent films, silicone crosspolymers with dissolving properties have been discovered to work quickly and completely to remove the film. The color containing film is removed instantly when applied to the lip, face or eyelids, eyelashes and then wiped off with a tissue. A single application completely removes the film and the color.

Currently commercial available lipstick and makeup removers, such as Cover Girl clean makeup remover for eyes and lips (contains mainly mineral oil) and Revlon Color Stay Eye & Lip Makeup Remover, (which itself contains a silicon crosspolymer-dimethicone crosspolymer-3—that does not have solvent properties) do not have sufficient solublizing power to instantly remove the lip coloring or other cosmetic completely with one application.

The organic oil that is used to add softening effect and emmoliency to the skin is octyl cocoate, or ethylhexyl palmitate, a higher quality version of octyl cocoate (higher distillate). It is preferred to use the octyl cocoate as the emollient, but many other esters can be used and include; hexadecyl laurate, hexyldecyl palmitate, hexydecyl stearate, cetyl palmitate, cetyl laurate, cetyl isononanoate, cetyl strarate, stearyl stearate, etc. Success with the octyl cocoate has been achieved with a single application.

Also included in the desired formulation of the invention are essential fatty acids (EFA's) and skin identical ceramides, which are composed of the same material as the interstitial fluid that is located between the cells of the skin and are among the main component of collagen.

Dimethicone/vinyldimethicone crosspolymer is the preferred silicone crosspolymer with solvent properties, but other silicone crosspolymers with solvent properties may be used, including: cetearyl dimethicone/vinyldimethicone crosspolymer, PEG 12 dimethicone crosspolymer, PEG 10 dimethicone crosspolymer, vinyldimethicone/methicone silsesquioxane crosspolymer (and blends of the above with dimethicone and/or cyclomethicones and the like).

The silicone crosspolymer is preferably mixed with cyclopentasiloxane to give the composition a smoother texture and reduce its viscosity. In addition to cyclopentasiloxanes, the following silicone based polymers have also been found effective at providing the proper consistency and include cycloheptasiloxane, cyclohexasiloxane, cyclomethicones, cyclotetrisiloxane, cyclotrisiloxane, dimethicone (and combinations of the former with dimethicone) and (combinations of all of the above without dimethicone).

I claim:

1. A cosmetic removal composition suitable for removing “permanent” or long lasting color cosmetics, comprising at least one silicone crosspolymer with solvent properties.

2. The cosmetic removal composition of claim 1, in which the silicone crosspolymers are selected from the following group of silicone crosspolymers with solvent properties: dimethicone/vinyl dimethicone cross polymer, cetearyl dimethicone/vinyl dimethicone crosspolymer, PEG 12 dimethicone crosspolymer, crosspolymer, and vinyldimethicone/methicone silsesquioxane crosspolymer.

3. The cosmetic removal composition of claim 1, in which the silicone crosspolymer is dimethicone/vinyl dimethicone cross polymer.

4. The cosmetic removal composition of claim 1, further comprising at least one cyclic dimethyl polysiloxane.

5. The cosmetic removal composition of claim 4, in which the cyclic dimethyl polysiloxanes are selected from the following group: cyclopentasiloxane, cycloheptasiloxane, cyclotetrasiloxane, cyclotrisiloxane, cyclomethicone, dimethicone.

6. The cosmetic removal composition of claim 1, further comprising a compatible and soluble emollient to form a semi-permanent film residue.

7. The cosmetic removal composition of claim 6, wherein said soluble emollient is octyl cocoate, an ester derived from coconut oil.

8. The cosmetic removal composition of claim 6, wherein said film residue includes a deposition of agents, such as essential fatty acids, interstitial fluids of the skin and ceramides of a collagen layer.

9. The cosmetic removal composition of claim 1, wherein said composition works quickly and completely to remove said permanent or long-lasting color cosmetics and wherein any colorants contained within said permanent or long-lasting color cosmetics are removed instantly when said cosmetic removal composition is applied to the lip, face, eyelids, eyelashes, and subsequently wiped with a tissue or other suitable wiping agent.

10. The cosmetic removal composition of claim 1, wherein a single application completely removes said permanent or long-lasting color cosmetics.

11. A cosmetic removal composition comprising a first silicone selected from the group consisting of
(i) Da wherein D is a dimethylsiloxy and a is 3-6 and the silicone is cyclomethicone;
(ii) MD_{b}D′_{c}D″;_{d}M wherein M is R³SiO_{0.5}, D is R²SiO_{1.0} where R is methyl and c and d are 0 and the silicone is dimethicone, and;
(iii) mixtures thereof; and a second silicone selected from the group consisting of a M_{g}Qₕ silicone resin and an MₑT_{f} silicone resin wherein M has the empirical formula R₃SiO_{0.5}, R¹RRSiO_{0.5}, or R¹R²RSiO_{0.5}, or R¹R²R³SiO_{0.5} wherein R is methyl and R¹, R₂, and R³ are each independently a C₁₋₆ straight or branched chain alkyl, or phenyl; T has the empirical formula RSiO_{1.5}, or R⁸SiO_{1.5} wherein R is methyl and R⁸ is a C₂₋₄₀ straight or branched chain alkyl which may be substituted with on or more hydroxyl or halogen radicals; and Q is SiO_{4/2}; and e is 1-1000; f is 1-1000; g is 1-1000; and h is 1-1000; and further comprising a synthetic polymer comprised of one or more monomers from acrylic acid, methacrylic acid, C₁₋₃₀ esters of acrylic acid, C₁₋₃₀ esters of methacrylic acid, vinyl pyrrolidone, methylene diisocyanate, vinyl acetate, vinyl isodecanoate, styrene, or olefms; and a silicone compatible organic oil comprising a paraffinic hydrocarbon; particulate matter, and wax.

12. The composition of claim 11, further comprising a soluble emollient such as octyl cocoate, an ester derived from coconut oil.

13. The composition of claim 11, further comprising one or more chemical sunscreen or sunblocking agents.

14. The composition of claim 11, further comprising particulate matters selected from one or more of the following: pigments, powders, or mixtures thereof.

15. The composition of claim 11, further comprising a cosmetically acceptable natural or synthetic wax.

16. The composition of claim 15 , wherein the wax is present at about 0.1-70% by weight of the total composition.

17. The composition of claim 11, additionally comprising a silicone compatible oil which is the reaction product of a fatty acid and glycerin.

18. The composition of claim 11 additionally comprising a silicone compatible organic oil which is a diester that is the reaction product of a dicarboxylic acid and an aliphatic or aromatic alcohol.

19. The composition of claim 11 additionally comprising a silicone compatible organic oil which is a monoester.

20. The composition of claim 11 additionally comprising a silicone compatible organic oil which is a triester.
